(19) United States
(12) Patent Application Publication
(10) Pub. No.: US 2023/0355888 A1
(43) Pub. Date: Nov. 9, 2023

(54) MEDICAL SYRINGE WITH NEEDLE GUARD

(71) Applicant: TRENTA2 S.R.L., Bozen (IT)

(72) Inventor: Dietrich GALLMETZER, Terlan (IT)

(73) Assignee: TRENTA2 S.R.L., Bozen (IT)

(21) Appl. No.: 18/026,039

(22) PCT Filed: Sep. 14, 2020
(86) PCT No.: PCT/EP2020/075655

§ 371 Date: Mar. 13, 2023

(51) Int. Cl. A61M 5/32

(52) U.S. Cl. CPC A61M 5/3202; A61M 5/3221; A61M 5/31591

(57) ABSTRACT

A syringe with needle protection with a plunger displaceable by means of an actuating plunger in the interior of a syringe body is designed for particularly effective delivery of the active substance with the lowest possible residual portion in the syringe that is not delivered. For this purpose, the plunger has a needle holder for receiving the syringe needle, the needle bearing of which is arranged on a retaining bracket, leaving a number of lateral inflow surfaces free.

The invention relates to a medical syringe with needle protection having a plunger which is displaceable in the interior of a syringe body via an actuating plunger.

To avoid or reduce the risk of contamination or injury after the use of medicinal syringes and in particular to avoid multiple use of syringe needles by different users, syringes with so-called retraction or retraction systems for the syringe needle are increasingly used. In such syringes, in particular medical syringes, also referred to as “syringe with (passive) needle protection”, the syringe needle is retracted into the syringe body after dispensing the active substance held in the syringe and is completely enclosed by the syringe body. Access to the syringe and thus a risk of injury, or even the risk of multiple use of the same needle, can thus be largely excluded.

Such syringes with passive needle protection are known, for example, from EP 1 284 769 B1, EP 0 720 491 B1, EP 0 680 347 B1 or EP 1 764 127 B1.

Generally speaking, in the case of medical syringes, and thus also in the case of syringes with needle protection of the type mentioned, there is a need to deliver the active substance contained therein as completely as possible when using the syringe and to minimize as far as possible the retention of unused residual quantities of the active substance within the syringe. The invention is now based on the task of specifying a syringe with needle protection of the above-mentioned type, with which this need is taken into account.

This task is solved according to the invention in that the plunger, which is displaceable in the interior of the syringe body, has a needle holder which is provided for receiving the syringe needle and whose needle bearing is arranged on a retaining bracket, leaving a number of lateral inflow surfaces free.

Advantageous embodiments of the invention can be taken from the subclaims and/or the following description of the figures.

The invention is based on the consideration that the complete delivery of the active substance as a result of the displacement of the plunger within the syringe housing towards its distal end is essentially limited by the so-called dead volume, i.e. pockets or dead spaces within the housing due to the geometry, from which the active substance can no longer flow out via the needle when the plunger is completely displaced. For the intended minimization of the active substance residues remaining in the syringe housing after use, these dead spaces should therefore be kept particularly small, and outflow channels should be provided in a targeted manner, through which the active substance can still flow towards the entry end of the needle even when the plunger is pushed far forward. This can be problematic, especially in syringes with needle protection, since in such systems the needle must be grasped when the plunger is far advanced and then retracted with the plunger into the interior of the syringe housing. The system for gripping and entraining the needle should therefore allow the active substance to flow as far as possible to the inner end of the needle, even when the plunger is advanced as far as possible. This is achieved by grasping the needle via a bracket that leaves free flow areas for the active substance in its lateral areas, through which the active substance can still flow to the needle.

Advantageously, the needle holder is made of plastic, preferably polypropylene. The polypropylene available under the designation “Bormed™” (HD810MO, ISO 10993 Information (Biocompatibility)) is particularly preferred as the basic material with regard to the expected required holding forces and the expected mechanical loads during intended use, but also with regard to approval-related requirements.

Advantageously, the needle holder is surrounded by a plunger jacket which is shaped in such a way that, when the needle holder is inserted, it leaves a number of inflow channels for the active substance at the side of the retaining clip. The piston jacket is preferably made of rubber.

In a particularly preferred embodiment, the syringe body is manufactured as a plastic part, preferably from cyclo-olefin polymer (COP). This material is characterized by high breaking strength and glass-like transparency. Moreover, it does not release any alkali ions, so that the risk of a pH value shift in the active ingredient stored is excluded. Thus, according to the invention, this material is considered and used as suitable even for the primary packaging of demanding medicines, in particular for sensitive biotechnologically produced active substances. In addition, this material is suitable for production by injection molding and thus for particularly precise dimensioning.

An embodiment of the invention is explained in more detail with reference to a drawing. Shown therein:

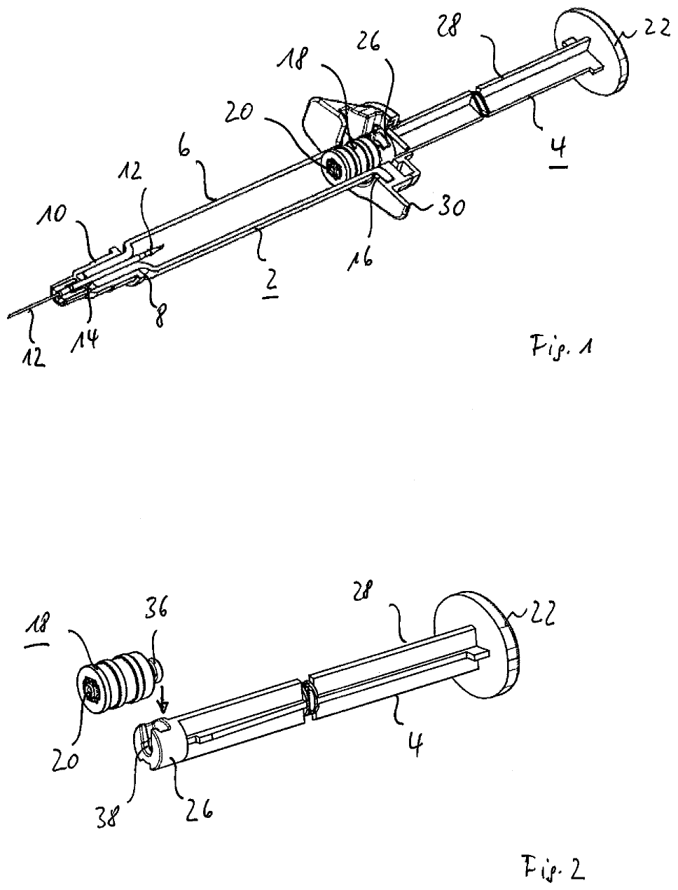

FIG. 1 a medical syringe with needle protection,

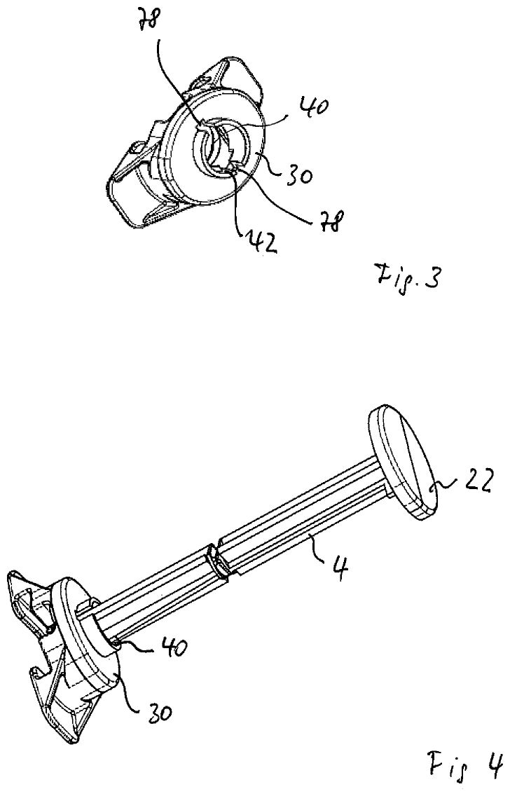

FIG. 2 a side view of an actuation unit of the syringe according to FIG. 1,

FIG. 3 a cover plate of the syringe according to FIG. 1,

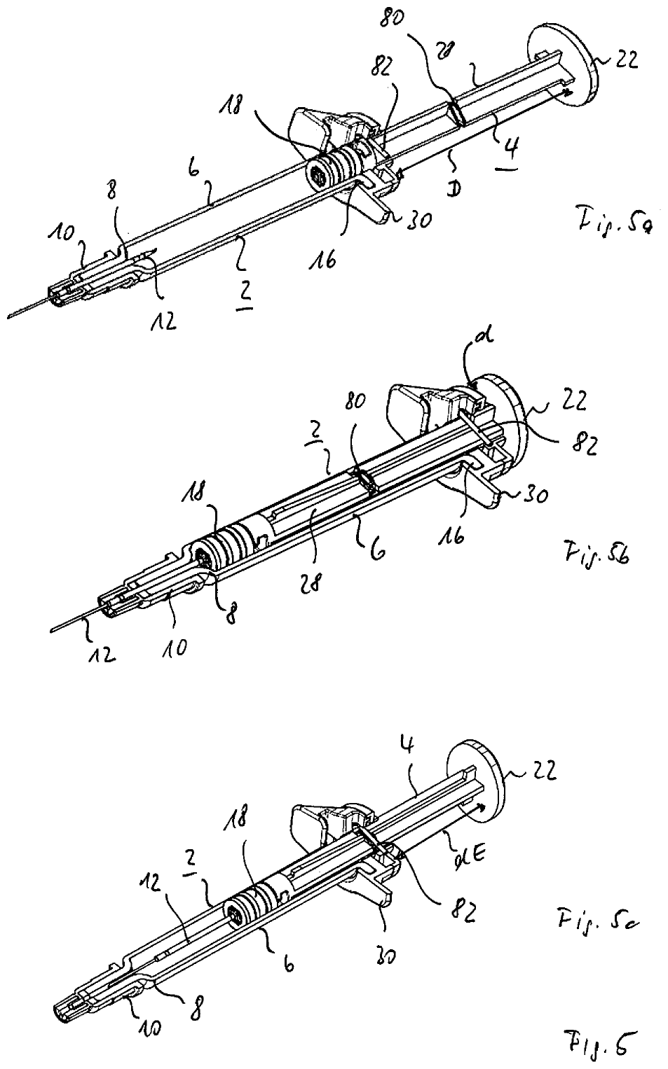

FIG. 4 the cover plate according to FIG. 3 with the actuating plunger attached,

FIG. 5 a sequence of partial sections of the syringe according to FIG. 1,

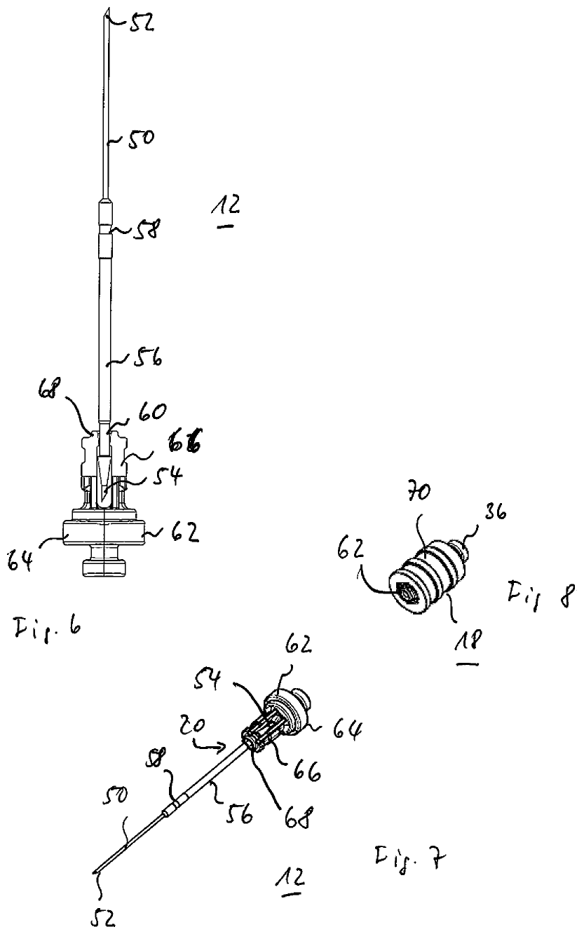

FIG. 6A side view of a needle inserted into a needle holder for use in the syringe according to FIG. 1,

FIG. 7 the needle according to FIG. 6 in perspective view,

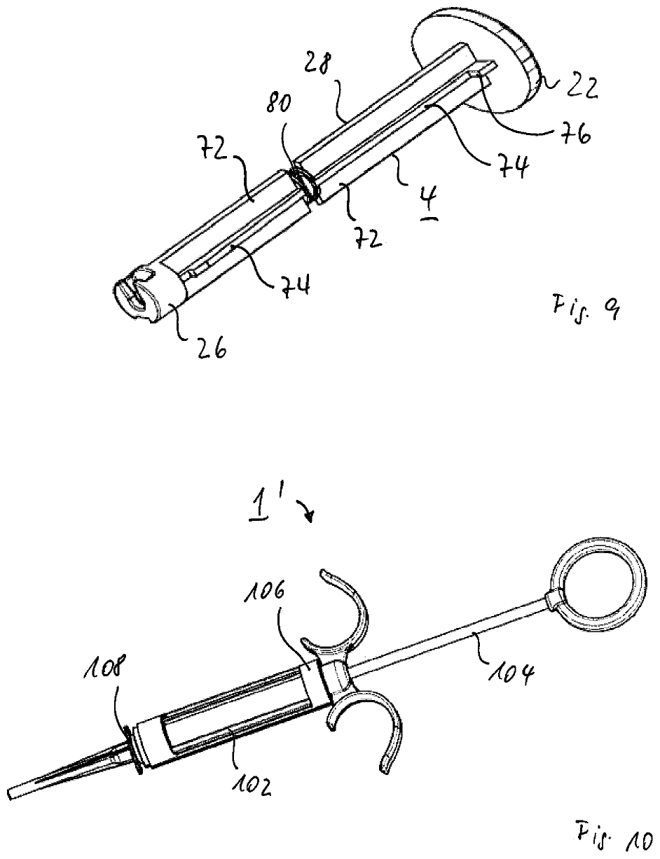

FIG. 8 a plunger of the syringe according to FIG. 1,

FIG. 9 an actuating plunger of the syringe according to FIG. 1,

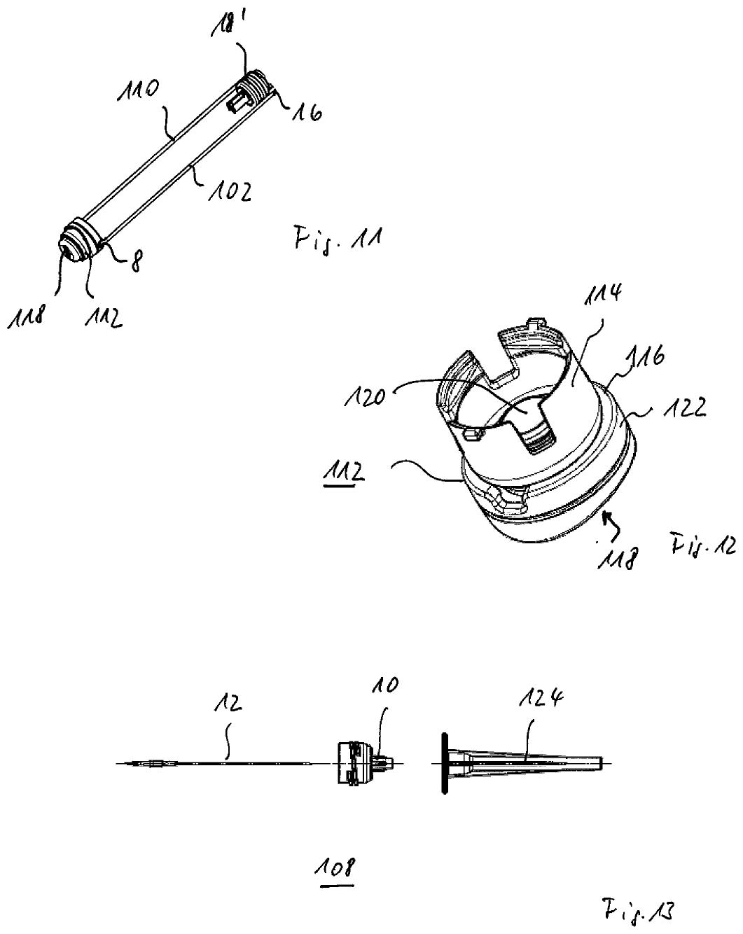

FIG. 10 an alternative embodiment of a medical syringe with needle protection,

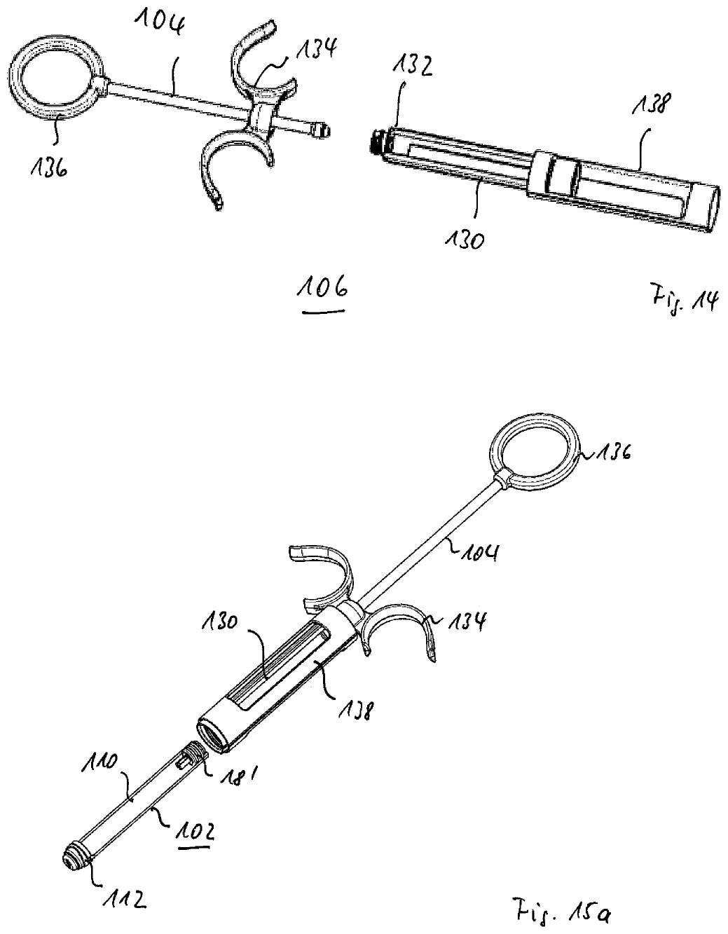

FIG. 11 a cartridge or cartridge unit of the syringe according to FIG. 10,

FIG. 12 a cap of the cartridge unit according to FIG. 11,

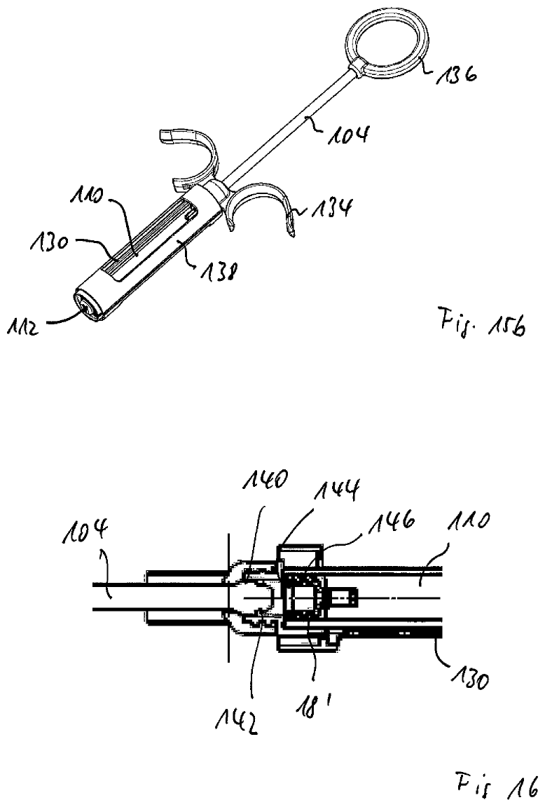

FIG. 13 an exploded view of the needle head of the syringe according to FIG. 10,

FIG. 14 a syringe frame of the syringe according to FIG. 10,

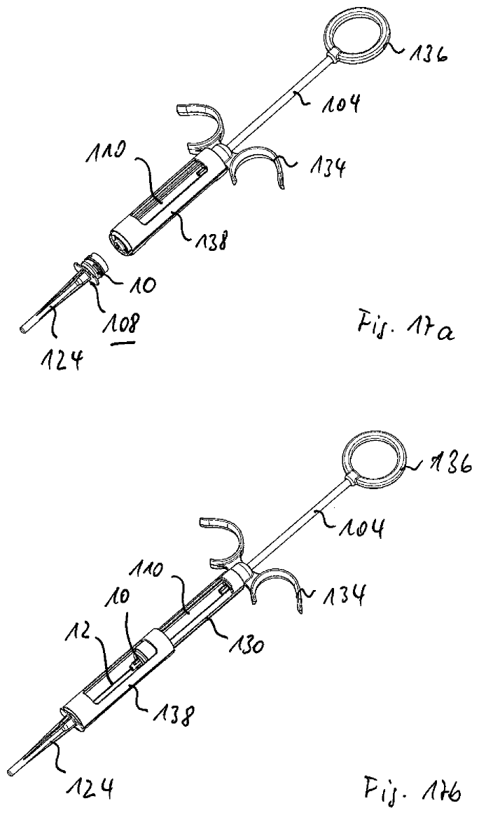

FIG. 15 the cartridge unit according to FIG. 11 immediately before (FIG. 15a) and after (FIG. 15b) insertion into the syringe frame according to FIG. 14,

FIG. 16 a longitudinal section of the connection area between the plunger and the actuating plunger of the syringe as shown in FIG. 10,

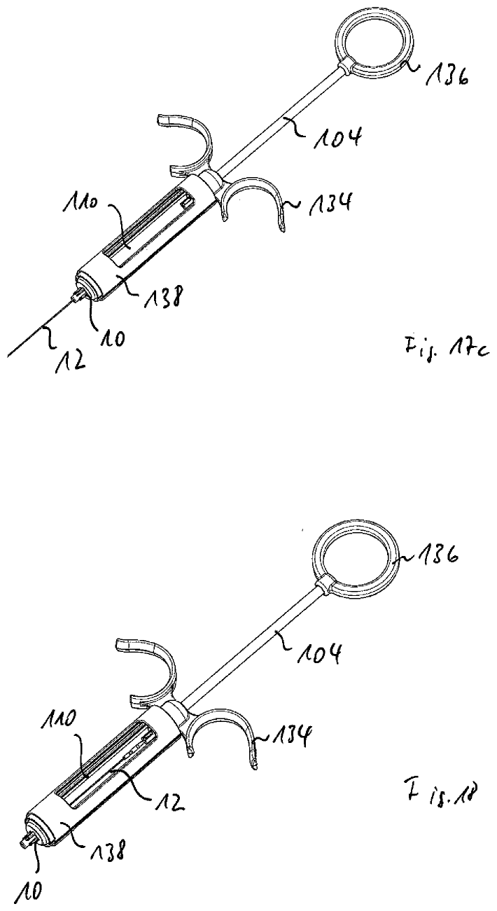

FIG. 17 the syringe as shown in FIG. 10 with the needle attached and exposed,

FIG. 18 the syringe according to FIG. 10 after application of the active substance and retraction of the needle,

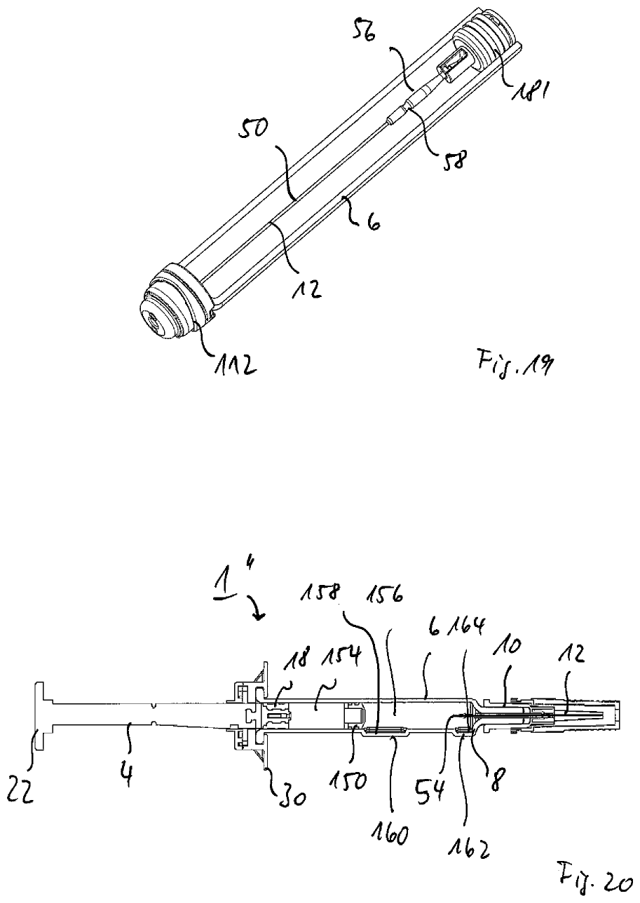

FIG. 19 the cartridge body of the syringe shown in FIG. 10 with the needle retracted therein, and

FIG. 20 another alternative embodiment of a medical syringe with needle protection and double chamber system in longitudinal section.

Identical parts are marked with the same reference signs in all figures.

The medical syringe 1 with needle retraction according to FIG. 1 essentially comprises two assemblies, namely on the one hand a cartridge unit 2 and on the other hand an actuating plunger 4 . In the embodiment example, the medical syringe 1 has a two-component design, with the two said assemblies forming separate components that can be connected to one another in the manner described below.

Alternatively, the syringe could also have a single-component design, in which case the two assemblies mentioned are connected to each other from the outset and the distinction between the two assemblies is merely functional.

The cartridge unit 2 forms the actual syringe and comprises a cylindrical or tubular hollow body 6 which forms a syringe housing and is intended to receive the medical active substance. A needle holder 10 is attached to the front or distal end 8 of the hollow body 6, in which the hollow needle 12 intended for injecting the active substance is mounted in a bearing sleeve 14. The needle holder 10 could be made in one piece with the hollow body 6 forming the syringe housing. In the embodiment example, however, the needle holder 10 is designed as a separate component in an embodiment considered to be independently inventive. The needle holder 10 is attached or attachable to the hollow body 6 forming the syringe housing or to the syringe cone, but could also be screwed on by means of a thread, for example a Luer thread.

With regard to the choice of material for the syringe housing or the hollow body 6 forming it, which is considered to be inventive in its own right, particular account is taken of high demands on the reliable temporary storage of the medical active substance together with a particularly high level of safety in handling the components. The syringe body 6, which is designed as a cylindrical hollow body, is made of the high-performance plastic cyclo-olefin polymer in an embodiment considered to be inventive. This material is characterized by high breaking strength and glass-like transparency. Moreover, it does not release any alkali ions, so that the risk of a pH shift in the active ingredient is excluded. The syringe body 6 is preferably manufactured by injection molding, whereby, among other things, the molding is carried out in such a way that possible dead volumes in the interior are kept particularly low.

To prevent injuries or the like, the needle 12 is surrounded by a removable protective cap, not shown in greater detail, which is removed before the syringe 1 is used. The rear or proximal end 16 of the hollow body 6 forming the syringe housing, on the other hand, can be closed by a piston 18 whose outer dimensions are precisely adapted to the inner contour of the hollow body 6 and which can be moved within the hollow body 6. In the state shown in FIG. 1, i.e. with the actuating plunger 4 connected to the cartridge or cartridge unit 2 and before the medicinal substance is taken up or dispensed, the medicinal substance is thus enclosed in the interior of the hollow body 6 closed at the end by the piston 18. On its end surface facing the interior of the hollow body 6, the piston 18 has a central receiving hole 20 for the needle 12.

The drive or actuating plunger 4 comprises a shaft 28 provided on one end with a plunger plate 22 and on the other end 24 opposite the plunger plate 22 with a coupling element 26 provided for connection to the piston 18. With its shaft 28 extending between the plunger plate 20 and the coupling element 26, the actuating plunger 4 is guided through a cover plate 30 in the assembled state of the components and is mounted in the latter so as to be displaceable in its longitudinal direction.

The piston 18 could be made in one piece with the actuating plunger 4. In the embodiment example, however, these components are designed separately. As can be seen from the illustration in FIG. 2, for connection to the shaft 28 of the actuating plunger 4, the piston 18 has a fastening pin 36 molded on at the end and provided with a circumferential groove. Corresponding to this and adapted in its dimensioning to it, the coupling element 26 molded onto the end of the shaft 28 is provided with an elongated hole 38 open on one side. The fastening pin 36 can be pushed laterally into this, so that the lateral edge of the elongated hole 38 engages in the circumferential groove of the fastening pin 36 and thus, viewed in the longitudinal direction, a form fit is created between these components.

The medical syringe 1 is provided with a needle protection in the form of a retraction system by the components and parts mentioned. The purpose of this is that after use of the syringe 1, i.e. after dispensing the active substance held in the hollow body 6 forming the syringe housing via the needle 12, the latter is drawn into the syringe housing in such a way that it is completely enclosed by the syringe housing. This is intended to keep unintentional contact with the used needle 12, for example by auxiliary or nursing personnel, and thus the risk of injury and contamination particularly low or, if possible, completely excluded.

For this purpose, the following procedure is basically intended for the use and handling of the components mentioned:

The syringe 1 could in principle be used with a pre-filled cartridge or cartridge unit 2. In this case, the cartridge or cartridge unit 2 filled with the active ingredient would be provided with the plunger 18 already inserted into the hollow body 6 and closing its interior. As a first step in use, the actuating plunger 4 is then connected at the end to the piston 18 in the manner shown in FIG. 2, and the system is ready to dispense the active substance.

In the embodiment example, however, it is intended that the syringe 1 is made ready for use when empty and is filled with the active substance by drawing it on. For this purpose, the actuating plunger 4 carrying the plunger 18 is first inserted into the receiving hole 40 provided for this purpose in the cover plate 30, so that the plunger 18 is introduced into the interior of the hollow body 6, as can be seen from the enlarged representation of the cover plate 30 from above at an angle according to FIG. 3 and of the cover plate 30 with the actuating plunger 4 attached from above at an angle according to FIG. 4. The receiving hole 40 with its outer edge 42 designed in the manner of a guide slot and the corresponding cross-section of the shaft 28 of the actuating plunger 4 are designed in such a way that the insertion takes place in a predetermined rotational alignment of the actuating plunger 4 relative to the hollow body 6 or its cover plate 30.

Subsequently, the actuating plunger 4 is pressed by the operator while maintaining this rotational orientation—this is achieved by a constant cross-sectional geometry of the shaft 28, seen in the longitudinal direction—so that the piston 18 moves inside the hollow body 6 towards its distal end 8. As soon as a predetermined end position is reached in which the piston 18 is still sufficiently far away from the needle 12 in the sense of the mechanism described below, this movement is stopped by a stop formed by the shaft 28. The stop is formed by an abrupt change or widening of the cross-section of the shaft 28 in the manner of a step or edge, as seen in the longitudinal direction, so that the shaft 28 cannot be guided further through the outer edge 42 of the receiving hole 40 in the longitudinal direction.

The cross-section of the shaft 28 and correspondingly the contour of the outer edge 42 of the receiving hole 40, which forms the guide slot, are designed in such a way that when the piston 18 reaches the said end position inside the hollow body 6, the rotational lock between the actuating plunger 4 and the cover plate 30 is cancelled at least to a certain extent and the plunger 4 can be rotated in the cover plate 30 about its longitudinal axis through a predetermined angle of rotation, preferably about 90°. This rotation achieves that, on the one hand, the previously present stop preventing further displacement of the plunger 18 in the direction of the distal end 8 is unlocked, so that the plunger 18 could now be pushed completely into the final position within the hollow body 6. On the other hand, however, this rotation also forms—again achieved by a suitable design of the cross-section of the shaft 28 and adapted thereto the contour of the outer edge 42—a new stop “upwards” or towards the proximal end 16 of the hollow body 6, so that the actuating plunger 4 and in particular the piston 18 attached thereto at the end cannot be pulled out of the hollow body 6 beyond an end point predetermined thereby.

In this position, the free end of the needle 12 is now inserted into an external reservoir of the active substance, and then the piston 18 is retracted in the hollow body 6 by means of the actuating plunger 4. The active substance is sucked in via the needle 12 and the interior of the hollow body 6 is thus filled.

After the syringe 1 has been made ready for use in one of the aforementioned ways, the needle 12 is positioned appropriately on the patient for the administration of the medical agent so that it pierces the patient's skin at a suitable point. The retaining circle of the needle 12 in the bearing sleeve 14 is thereby predetermined, in particular by suitable dimensioning of the components and/or the choice of material pairing, in such a way that the needle 12 remains securely in its position in the bearing sleeve 14 when the operator pierces the needle 12 through the patient's skin by handling it on the hollow body 6.

The actuating plunger 4 is then pressed by the operator so that the piston 18 moves inside the hollow body 6 towards its distal end 8, thereby feeding the medicinal agent to the needle 12 and dispensing it via the latter. Shortly before complete dispensing of the active substance, i.e. shortly before complete emptying of the interior of the hollow body 6, the piston 18 reaches the end of the needle 12 projecting into the interior in the vicinity of the distal end 8 of the hollow body 6, so that the needle 12 penetrates the receiving hole 20 provided for this purpose during further movement. After complete dispensing of the active substance, the piston 18 then reaches its end position directly at the distal end 8 of the hollow body 6 and thereby encloses the part of the hollow needle 12 projecting into the receiving hole 20. This insertion of the corresponding part of the hollow needle 12 into the receiving hole 20 and the connection of the needle 12 with the piston 18 thereby achieved is also referred to herein as “connecting”.

After the active substance has been administered and in particular after the hollow body 6 has been completely emptied, the operator retracts the pusher plate 22 and with it the actuating plunger 4 as a whole. The piston 18, which is connected to the shaft 28 of the actuating plunger 4 via the coupling element 26, is thus also carried along and pulled within the hollow body 6 away from the distal end 8 towards the proximal end 16. In turn, it takes the enclosed needle 12 with it and pulls it into the hollow body 6 so that it is completely positioned inside the hollow body 6 in the final state.

For better clarification, this sequence is shown in the sequence of partial sections in FIG. 5. FIG. 5a shows the syringe 1 before administration of the active substance held in the interior of the hollow body 6. The plunger 18 guided in the interior of the hollow body 6 is located at its stop on the cover plate 30 arranged at the proximal end 16 of the hollow body 6. The actuating plunger 4 is accordingly fully extended, and the plunger plate 22 is located at the maximum distance D from the cover plate 30. The hollow needle 12 held in the needle holder 10 is extended ready for use in this state.

Starting from this state, the active substance is dispensed—in particular when the needle 12 is inserted into the patient's skin—by moving the plunger 18 from its starting position shown in FIG. 5a to the end position shown in FIG. 5b immediately adjacent to the distal end 8 of the hollow body 6. For this purpose, the actuating plunger 4 is pushed into the hollow body 6 up to the end position in which the pusher plate 22 assumes the minimum distance d from the coupling plate 30. The hollow needle 12 held in the needle holder 10 is still extended in this state, but has already entered the receiving opening 20 of the piston 18 and is enclosed by the piston 18 with its end projecting into the interior of the hollow body 6 and is mechanically connected to it.

After the piston 18 or the actuating plunger 4 has reached the end position shown in FIG. 5b and the active substance has thus been completely dispensed, the actuating plunger 4 is retracted again by the operator to the end position shown in FIG. 5c. In this end position, the pusher plate 22 is in a position with the end distance dE from the cover plate 30. Correspondingly, the plunger 18 has moved along in the process, so that in this position it is in a central position within the hollow body 6. The hollow needle 12 has been carried along by the piston 18 and is now completely inside the hollow body 6.

In order to achieve the mode of operation explained above in a particularly reliable and advantageous manner in several respects, the components are specifically designed in various details, whereby the embodiments described below are each considered to be both independently inventive and inventive in any combination with one another.

For example, the hollow needle 12 is independently inventive according to the following description. As can be seen from the enlarged representation in FIG. 6 (side view) and FIG. 7 (perspective view), the hollow needle 12 comprises, in an independently inventive design, a needle tube 50 made of metal, which forms a needle tip 52, 54 at each of its two ends. The material for the needle tube 50 is preferably selected with regard to common requirements for medical applications, whereby a stainless material which can also be used for standard needles is particularly preferred. In its middle length range, the needle tube 50 is sheathed and surrounded by a plastic sheath 56. The material for the plastic sheath 56 is preferably a polyamide (PA12), most preferably the one commercially available under the designation Vestamid Care ML 17. The plastic sheath 56 is sprayed onto the needle tube 50 in a particularly preferred embodiment, which is also considered to be independently inventive, after the latter has been subjected to a plasma pretreatment in the manner of a surface activation. In this way, a particularly good adhesion of the plastic forming the plastic sheath 56 to the needle 50 can be achieved. Two retaining grooves 58, 60 are formed in the plastic jacket 56 to enable the above-described mode of operation.

The first retaining groove 58 is provided for temporarily fixing the needle 12 in the bearing sleeve 14 of the needle holder 10. For this purpose, an associated circumferential locking lip is provided inside the bearing sleeve 14, which engages in the holding groove 58 when the needle 12 is mounted and properly inserted into the bearing sleeve 14 and fixes it in the longitudinal direction. According to an embodiment which is basically considered to be independently inventive, the dimensions of the retaining groove 58 and the detent lip are advantageously selected in such a way that, taking into account the deformability of the material of the plastic sheath 56 and/or any adhesive force due to the material pairing of the material of the plastic sheath 56 and the material of the bearing sleeve 14 surrounding it, the retaining or breakaway force of the needle 12 thus engaged in the longitudinal direction is, on the one hand, sufficiently large, so that the needle 12 can be inserted into the patient's skin in accordance with the procedure described above, but on the other hand is also sufficiently small so that the described retraction movement of the needle 12 towards the interior of the hollow body 6 can be carried out. If necessary, the profile of the holding groove 58 can also be designed accordingly asymmetrically, with a comparatively steep flank angle on its side facing the tip 54 facing the interior and a comparatively flat flank angle on its side facing the exposed tip 52.

The second retaining groove 60, on the other hand, is provided for a corresponding catch in the piston 18. In FIGS. 6, 7 the needle 12 is shown in the inserted state in the piston 18. The piston 18 is in turn made in several parts and comprises the needle holder 62 according to the invention, which is also shown in FIGS. 6, 7. The needle holder 62 is made as a plastic part and consists, in the example, of the polypropylene available under the designation “Bormed™” (HD810MO, ISO 10993 Information (Biocompatibility)) with regard to the required holding forces and the mechanical loads expected during intended use, but also with regard to approval-related requirements.

As can be seen from the illustration in FIG. 6, and particularly well from the perspective view in FIG. 7, the needle holder 62 comprises a retaining bracket 66 molded onto a base body 64, which supports the actual needle bearing 68 forming the receiving hole 20. The needle bearing 68, analogous to the bearing sleeve 14 described above, is provided on the inside with an associated circumferential latching lip which, when the needle 12 is inserted into the needle bearing 68, engages in the second retaining groove 60 and fixes it in the longitudinal direction. In accordance with an embodiment which is also regarded in principle as independently inventive, the dimensions of the retaining groove 60 and of the detent groove in the needle bearing 68 associated therewith are advantageously selected in such a way that, taking into account the deformability of the needle 12, the deformability of the needle bearing 68 is not impaired, that, taking into account the deformability of the material of the plastic jacket 56 and/or a possible adhesive force due to the material pairing of the material of the plastic jacket 56 and the material of the needle bearing 68 surrounding it, the holding or breakaway force of the needle 12 thus engaged in the longitudinal direction is greater than the corresponding holding or breakaway force of the holding groove 58 in the bearing sleeve 14, so that during the retraction movement of the piston 18 the needle 12 is carried along by the latter towards the interior of the hollow body 6.

The design of the retaining clip 66 also creates a free space inside the plunger 18 which, in the final phase of dispensing the active substance, when the needle tip 54 has already penetrated the receiving hole 20 and is therefore no longer readily accessible to the active substance, allows the active substance to flow into the needle tube 50 via the needle tip 54 in the manner of a bypass. The inflow can take place on both sides of the retaining clip 66 into the free space.

As already explained, in a further embodiment, which is also considered to be independently inventive, the piston 18 is made of several parts. As can be seen in the enlarged view according to FIG. 8, in addition to the already described needle holder 62 for the needle 12, a piston jacket 70 surrounding the needle 12 is provided. The piston skirt 70 is made of conventional rubber, particularly preferably of the material available from Kreiburg under the designation TM4RST (MC/RS Series), taking into account approval-related requirements. The piston jacket 70 is shaped in such a way that, when the needle holder 62 is inserted, it leaves free inflow channels for the active substance on both sides of the retaining clip 66, so that, in the sense of avoiding or minimizing the dead volume, the bypass for the desired zero volume output is formed.

In order to provide the stops required to carry out the sequence described above when operating the syringe 1 to specify the respective intended end positions of piston 18 or shaft 28 within the hollow body 6, a specific design of the shaft 28 of the actuating plunger 4 is provided which is also considered to be independently inventive. For clarification, this is shown enlarged in FIG. 9. As can be seen from this illustration, the shaft 28 is essentially formed by two crossed shaft ribs 72, 74 forming a cross in cross-section. The upper edges of the shaft ribs 72, 74, as seen in the longitudinal direction of the actuating plunger 4, run essentially in a straight line and over large parts of the total length of the actuating plunger 4 essentially parallel to its longitudinal direction. However, at a position relatively close to the pusher plate 22, the shaft rib 74 has a projection so that an edge 76 is formed here. During the inward movement of the actuating plunger 4 into the hollow body 6, which is initially intended for filling the syringe 1, the edge 76 strikes the cover plate 30 and thus limits this inward movement.

After the subsequently provided rotational movement of the actuating plunger 4 about its longitudinal axis through the preferably provided angle of rotation of about 90°, the first shaft rib comprising the edge 76 is rotationally brought into overlap with a guide groove 78 formed by the outer contour 42 of the receiving hole 40, so that in this orientation the actuating plunger 4 can be displaced even further towards the distal end 8 of the hollow body 6.

Furthermore, a notch 80 is also provided in the shaft ribs 72, 74, into which a securing spring 82 arranged in the cover plate 30 can engage when positioned appropriately. This serves to fix the assumed position after retraction of the needle 12 into the interior of the hollow body 6, so that the needle 12 remains secured there.

An alternative medical syringe 1′ with needle retraction, considered to be independently inventive, is shown in FIGS. 10 ff. This medical syringe 1′ is intended in particular for cartridges pre-filled with active substance and comprises essentially three subassemblies, namely a cartridge unit 102, a frame 106 provided with an actuating plunger 104 and a needle head 108. In this embodiment example, the cartridge unit 102 shown enlarged in FIG. 11 is pre-filled with the active substance and comprises the actual cartridge body 110, which is designed as a cylindrical hollow body and is closed at its proximal end 16 with the plunger 18′. At the distal end 8, however, it is closed by a clipped-on or attached cap 112, as shown in FIG. 12.

The cap 112 shown in FIG. 12, which is considered to be independently inventive, comprises a cover 116 provided with a circumferential side skirt 114 which is provided as a clip for connection to the edge of the cartridge body 110. This comprises a central receiving hole 118 for the hollow needle 12, and it is provided on the inside with a preferably injection-molded seal 120 of suitably selected material, in particular TPE. The side skirt 114 is surrounded by a slidable sleeve 122. After being clipped or sprung onto the edge of the cartridge body 110, the sleeve 122 can be slid over the side skirt 114, which is provided with latching hooks, so that the hooking is fixed and the connection is thus secured.

The needle head 108 shown in exploded view in FIG. 13 comprises the needle holder 10, the hollow needle 12 and a needle protection cap 124. In addition, a seal 126 is provided to maintain sterility.

As can be seen from FIG. 14, the syringe frame 106 comprises a preferably cylindrical or semi-cylindrical chamber body 130 which is open at the ends and is provided for receiving the cartridge unit 102 and at the proximal end 132 of which, in addition to a finger rest 134, the actuating plunger 104, which in this embodiment example is provided at the ends with a finger tab 136, is arranged. The chamber body 130 is surrounded by a needle protection sleeve 138 that can be moved in the longitudinal direction.

With regard to the above-mentioned design goals of a simple but high-quality construction that meets high safety requirements, the finger rest 134 and the finger tab 136 are made of polyphenylsulphone (PPSU), a highly technical special plastic that can be sterilized almost as often as required with high mechanical stability and resilience and is considered a metal substitute for medical products. The actuating plunger 104, on the other hand, is made of chrome steel. In its entirety, the handle 2 can thus be sterilized many times and repeatedly, in particular due to its choice of material, and can thus be reused many times.

When using the medical syringe 1′, the filled and end-sealed cartridge body 110 is first fully inserted through its open end into the chamber body 130, as shown in FIG. 15. FIG. 15a shows the components immediately before, FIG. 15b the components immediately after this insertion. In this embodiment example, the piston 18′ is thereby designed for a particularly simple coupling with the actuating plunger 104 in a manner considered to be independently inventive. As can be seen from the illustration in longitudinal section according to FIG. 16, the end of the actuating plunger 104 has a thickening 140 with a circumferential locking groove 142. Correspondingly, the piston 18′ in this embodiment is provided with a receiving channel 144 into which the thickening 140 can be inserted. Corresponding to the detent groove 142, the receiving channel 144 is provided with a circumferential detent lip 146 which engages with the detent groove 142 when fully inserted. This connects the piston 18′ to the actuating plunger 4′.

FIG. 17 shows how the medical syringe 1′is subsequently made ready for use. For this purpose, the needle head 108 is first attached after the cartridge body 110 has been inserted into the chamber body 130, as shown in FIG. 17a. This can be done in a particularly simple manner by screwing on; for this purpose, preferably the needle holder 10 in this embodiment example is provided on the outside with a preferably double-threaded Luer thread which cooperates with a corresponding internal thread in the end region of the chamber body 130. In this assembly, the internal needle tip 54 pierces the seal 120 in the receiving hole 118 and thus protrudes into the interior of the cartridge body 110. Then, as shown in FIG. 17b, the needle guard 138 is moved towards the needle 12 so that it lifts off the needle guard 124. This is thus removed without the user running the risk of pricking themselves, and then the needle guard 138 is retracted so that the needle 12 is now ready for use and exposed, shown in FIG. 17c.

The active substance can then be administered by pressing the actuating plunger 104, whereby, analogous to the mode of operation already described above, the “connecting” of the needle 12 with the plunger 18′ and the insertion of the corresponding partial area of the hollow needle 12 into the receiving hole 20 of the plunger 18′ takes place after complete delivery of the active substance. The operator then retracts the actuating plunger 104 again. Thus, also in this case, the piston 18′ connected to the actuating plunger 104 and, with it, the encased needle 12 are carried along, so that the latter is completely drawn into the cartridge body 110. As can be seen from the representation of this state in FIG. 18, the needle holder 10 can then be unscrewed again. Subsequently, by pulling the actuating plunger 104, the user can release its engagement with the plunger 18′ again, so that the cartridge body 110, which now completely encloses the needle 12, can be removed from the chamber body 130.

The cartridge body 110, which is thus ready for safe disposal and completely encloses the needle 12, is shown in FIG. 19. After its removal from the chamber body 130, the frame 106, designed as a reusable system and intended for multiple use, can be put to further use.

An alternative embodiment of a medical syringe 1″ with passive needle protection of the type mentioned, which is also considered to be independently inventive, is shown in longitudinal section in FIG. 20. FIG. 20 shows the medical syringe 1″ before the active substance is dispensed. In this alternative embodiment, the medical syringe 1″ is equipped with a double chamber or double piston system. In an otherwise largely identical design to the aforementioned embodiment, the medical syringe 1″ has, in addition to the plunger 18, a further, upstream plunger 150 inside the hollow body 6. A first active substance chamber 154 is formed by the space between the piston 18 and the further piston 150, and the second active substance chamber 156 is then located between the further piston 150 and the apical end 8 of the hollow body 6.

Such a double-chamber system is particularly used for liquid and/or lyophilized (freeze-dried) or powdered medicines that need to be dissolved before administration. Such double-chamber systems are thus a particularly suitable solution for lyophilized/liquid or liquid/liquid drug combinations. The system offers a variety of advantages for sensitive injectable medications.

The solvent, for example, is kept in the first active substance chamber 154, whereas the actual active substance, e.g. freeze-dried or powdered, is located in the second active substance chamber 156. When the active ingredient is administered, the solvent provided in the first active ingredient chamber 154 is first introduced into the second active ingredient chamber 156 by actuating the actuating plunger 4 via a bypass channel 160 arranged in the housing wall of the hollow body 6 and formed by a molding 158 in the housing jacket, past the additional plunger 150. There it dissolves the active substance held there so that it is ready for administration. Subsequently, by further actuation of the actuating plunger 4, the plunger 18 is moved up to the stop against the plunger 150, and then the plungers 18, 150 are moved further together, so that the meanwhile dissolved active substance located in the second active substance chamber 156 is dispensed via the needle 12.

As soon as the further plunger 150 reaches the needle 12 and the latter penetrates the plunger body, the remaining active substance is dispensed and then the retraction system is triggered according to the mode of operation described above. The reduction of the dead volume in the end region of the hollow body 6 is thereby made possible by a further formation 164 forming a bypass channel 162, via which the residual amount of active substance can flow towards the end 54 of the needle 12 in a manner comparable to the embodiment already described above.

In this alternative embodiment of the medical syringe 1″, the needle holder 10 could also be made in one piece with the hollow body 6 forming the syringe housing. However, in the present embodiment example, which is considered to be independently inventive, the needle holder 10 is designed as a separate component and is plugged or attachable to the hollow body 6 forming the syringe housing or to the syringe cone. Alternatively, the needle holder 10 could also be designed to be screwed onto the hollow body 6 by means of a thread, in particular a Luer thread in this case.

### LIST OF REFERENCE SIGNS

1, 1′, 1″ Medical syringe
2 Cartridge or cartridge unit
4 Actuating plunger
6 Hollow body
8 Distal end
10 Needle holder
12 Hollow needle
14 Bearing sleeve
16 Proximal end
18 Piston
20 Receiving hole
22 Pusher plate
24 End
26 Dome element
28 Shaft
30 Lid plate
36 Fixing pin
38 Long hole
40 Receiving hole
42 Outer rim
50 needle tube
52, 54 Needle point
56 Plastic sheath
58, 60 Holding groove
62 Needle holder
64 Body
66 Holding bracket
68 Needle bearing
70 Piston skirt
72, 74 Shaft rib
76 Edge
78 Guide groove
80 Notch
82 Safety spring
102 Cartridge or cartridge unit
104 Operating plunger
106 Frame
108 Needle head
110 Cartridge body
112 Cap
114 Side Skirt
116 Lid
118 Receiving hole
120 Seal
122 Sleeve
124 Needle protection cap
126 Sealing
130 Chamber body
132 proximal end
134 Finger rest
136 Finger flap
138 Needle protection sleeve
140 Thickening
142 Locking groove
144 Receiving channel
146 Snap lip
150 Pistons
154, 156 Active substance chamber
158, 164 Molding
160, 162 Bypass channel
D maximum distance
d minimum distance
dE end distance

1. A syringe with needle protection designed in the manner of a retraction system, the syringe comprising:
a syringe housing;
a syringe needle that retracts into the syringe housing after the active substance held in the syringe housing has been dispensed; and
a plunger which can be displaced in an interior of a syringe body of the syringe by means of an actuating plunger in the interior of the syringe body, which plunger has a needle holder provided for receiving the syringe needle during retraction,
wherein:
the needle holder includes a retaining bracket which is integrally formed on a base body and which carries the actual needle bearing forming a receiving hole for the needle, and
the needle holder is arranged in a piston jacket of a piston of the syringe, which is shaped in such a way that, when the needle holder is inserted, the piston jacket leaves free inflow channels for the active substance on both sides of the holding bracket in such a way that a free space is created inside the piston, which, in the final phase of the application of the active substance, in which the needle tip of the needle has already penetrated into the receiving hole and is thus no longer readily accessible to the active substance, allows the active substance to flow into the needle tube via the needle tip in the manner of a bypass.

2. The syringe according to claim 1, wherein the needle holder is manufactured as a plastic part.

3. (canceled)

4. The syringe according to claim 1, wherein the plunger jacket is formed from rubber.

5. The syringe according to claim 1, wherein the syringe body is manufactured as a plastic part.

6. The syringe according to claim 2, wherein the plunger jacket is formed from rubber.

7. The syringe according to claim 2, wherein the syringe body is manufactured as a plastic part.

8. The syringe according to claim 7, wherein the syringe body is formed from cyclo-olefin polymer (COP).

9. The syringe according to claim 4, wherein the syringe body is manufactured as a plastic part.

10. The syringe according to claim 9, wherein the syringe body is formed from cyclo-olefin polymer (COP).

11. The syringe according to claim 2, wherein the needle holder is formed from polypropylene.
